(19) United States
(12) Patent Application Publication
(10) Pub. No.: US 2021/0062166 A1
(43) Pub. Date: Mar. 4, 2021

(54) INTRACELLULAR DELIVERY OF TARGET-SILENCING PROTEIN

(71) Applicant: Evox Therapeutics Ltd, Oxford (GB)

(72) Inventors: Joel NORDIN, Stockholm (SE); Justin HEAN, Oxford (GB); Per LUNDIN, Oxford (GB)

(21) Appl. No.: 16/960,351

(22) PCT Filed: Jan. 18, 2019
(86) PCT No.: PCT/EP2019/051238

§ 371 Date: Jul. 7, 2020

(30) Foreign Application Priority Data
Jan. 19, 2018 (GB) 1800921.7

(51) Int. Cl. C12N 9/10; C07K 16/18; C07K 14/705; A61K 9/10; A61K 38/45; A61K 39/395

(52) U.S. Cl. CPC C12N 9/104; C12Y 203/02; C07K 16/18; C07K 14/70596; A61K 2039/505; A61K 38/45; A61K 39/3955; C07K 2319/055; A61K 9/10

(57) ABSTRACT

The present invention relates to extracellular vesicle (EV)-mediated delivery of protein-based therapeutics. More specifically, the invention relates to delivery of complex polypeptide-based agents which typically bind to target proteins extracellularly, intracellularly, or in the cell membrane.

### TECHNICAL FIELD

The present invention pertains to the delivery of agents which induce protein-specific protein-mediated degradation.

### BACKGROUND ART

Protein biologics as therapeutics have seen increased implementation in the treatment and prevention of a wide range of diseases, including cancer, genetic disorders and autoimmune diseases. A majority of these are today's blockbuster drugs and as a result of their extracellular therapeutic activity they are very often administered without any delivery agent. The most common mode of action of protein biologics (such as recombinantly produced monoclonal antibodies) is decoying its extracellular target, optionally followed by enhancing immune system activation, for instance by antibody-dependent cellular cytotoxicity (ADCC).

Importantly, the intracellular milieu is highly restricted to large protein-based biologics, which means that the decoying (e.g. silencing) of intracellular target proteins is out of scope for most protein therapeutics. Considerable effort has however been made in developing delivery vectors for various forms of RNA therapeutics (e.g. antisense oligonucleotides, short interfering RNAs, splice-switching RNAs, etc.) that interfere with gene expression or translation, thereby resulting in silencing of particular genes, and, importantly, their corresponding proteins. However, RNA therapeutics do not deal with existing populations of target proteins, and show delayed efficacy against long lived proteins or proteins otherwise resistant to depletion. As such, RNA therapeutics may be less suitable for acute conditions where fast-acting treatments are needed, for instance in organ failure, stroke, or infectious diseases. The use of small molecule inhibitors, which readily cross the cellular membrane, has shown efficacy in the immediate depletion/deactivation of target proteins, however these methods are also fraught with non-specific effects. U.S. Pat. No. 8,530,636, WO2012/078559, WO2010/125620 teaches that attempts at posttranslational protein depletion have been made, however these approaches are specific to particular target proteins and lack the general applicability required of a novel drug modality or class. Until recently, a broadly applicable means to deplete target-protein populations has thus not been available. TRIM-Away (Clift et al., Cell, 2018) is one recent system that has the capacity to deplete a specific protein population in a post-translational protein-dependent manner. The system is broadly applicable to large range of proteins within a cell. The TRIM-Away system relies on the ubiquitination of target proteins through the action of a ubiquitin ligase. More specifically, TRIM21 (a ubiquitin ligase) interacts specifically with the Fc domain of a protein-targeting antibody; this interaction brings the target protein within the appropriate ubiquitination by TRIM21, which stimulates the degradation of the target protein. The TRIM-Away system has been limited to in vitro applications, as in vivo delivery of TRIM21 as well as the appropriate antibody targeting the protein of interest is a considerable obstacle in an in vivo setting. The bi-component nature of the TRIM system (and any other system based on the action of a suitable ubiquitin ligase) also means that conventional delivery vehicles such as lipid nanoparticles (LNPs), polymeric delivery systems, and/or liposomes are unlikely to be effective, as the TRIM system requires delivery of therapeutically relevant doses of the ubiquitin ligase and in most cases also a suitable antibody against the target protein.

### SUMMARY OF THE INVENTION

It is hence an object of the present invention to overcome the above-identified issues surrounding intracellular bioactive delivery of large biomolecular ubiquitin ligase-based protein degradation technology. The present invention addresses numerous obstacles relating to production and delivery of complex protein degradation systems, for instance the issue of delivering two separate large proteins in one delivery vehicle; to enable intracellular delivery of protein biologics such as the ubiquitin ligase enzyme and the antibody which mediates the target protein degradation; to enable targeted delivery of the ubiquitin ligase-based protein degradation system; and to enable scalable manufacturing and purification of ubiquitin ligase optionally combined with a suitable antibody to support therapeutic use in vivo of this technology.

The inventors of the present invention have surprisingly discovered that extracellular vesicles (EVs), for instance exosomes, can be engineered to deliver ubiquitin ligases to enable ubiquitin-mediated target protein degradation. The present invention thus relates to engineered EVs comprising at least a ubiquitin ligase but optionally also an antibody binding to a target protein to be degraded. Consequently, the present invention provides for a highly modifiable, targetable, and modular delivery vehicle for a very complex biological system undeliverable by other means.

In a first aspect, the present invention relates to an EV comprising a ubiquitin ligase. The ubiquitin ligase may preferably be an E3 ubiquitin ligase, and even more preferably a TRIM21 ligase or a domain or region thereof. In preferred embodiments, the ubiquitin ligase is fused to an exosomal protein in order to load the ligases into the EVs at a high number of enzyme per EV. Furthermore, in preferred embodiments, the EV will preferentially comprise an antibody, preferentially an antibody against an intracellular target.

In a second aspect, the present invention provides for a method of degrading a target protein, comprising the steps of (i) providing a target antigen bound by an antibody and (ii) using an EV to deliver a ubiquitin ligase into proximity of the antibody. In a preferred embodiment, the antibody, which is intended to bind to a target antigen present on a protein which is targeted for degradation, is also delivered by an EV, which may optionally be the same EV.

In a further aspect, the present invention relates to a polypeptide construct comprising an exosomal protein and a ubiquitin ligase, and in yet other aspects the invention relates to a polynucleotide construct encoding for such a polypeptide construct, and to a vector comprising the polynucleotide construct.

In yet another aspect, the present invention relates to a method for producing EVs as described herein. Such methods for EV production typically comprise the steps of introducing into an EV-producing cell at least one polynucleotide construct and expressing in the EV-producing cell at least one polypeptide construct encoded for by the at least one polynucleotide construct. Finally, EVs produced by the EV-producing cells are obtained via conventional methods for EV isolation and/or purification.

In a further aspect, the present invention relates to a pharmaceutical composition comprising a population of EVs as per the invention, together with a pharmaceutically acceptable excipient or diluent. The pharmaceutical composition may further comprise at least one antibody. Naturally, the EVs, the population of EVs, and/or the pharmaceutical composition of the present invention may be used in medicine, for instance in the treatment of cancer, autoimmune or inflammatory diseases, cardiovascular diseases and/or the like.

### BRIEF DESCRIPTION OF THE FIGURES

FIG. 1 illustrates various embodiments according to the present invention. From top to bottom, the drawings illustrate (i) an EV engineered to comprise multiple copies of ubiquitin ligases (UL), (ii) an EV engineered to comprise a fusion polypeptide between an exosomal protein (EP) and a ubiquitin ligase, where (a) the exosomal protein is a membrane protein, e.g. CD63 (b) the exosomal protein is a soluble protein, e.g. syntenin (c) the exosomal protein is released from the ubiquitin ligase, (iii) an EV engineered to comprise multiple copies of ubiquitin ligases in the lumen, combined with antibodies (AB) attached to their surface, which may be achieved through the expression of a fusion protein between an exosomal protein and an Fc-binding polypeptide (FBP), and (iv) an EV engineered to comprise a fusion polypeptide between a ubiquitin ligase and an exosomal protein, wherein the ubiquitin ligase, which may advantageously be TRIM21, is binding to an antibody against a target protein.

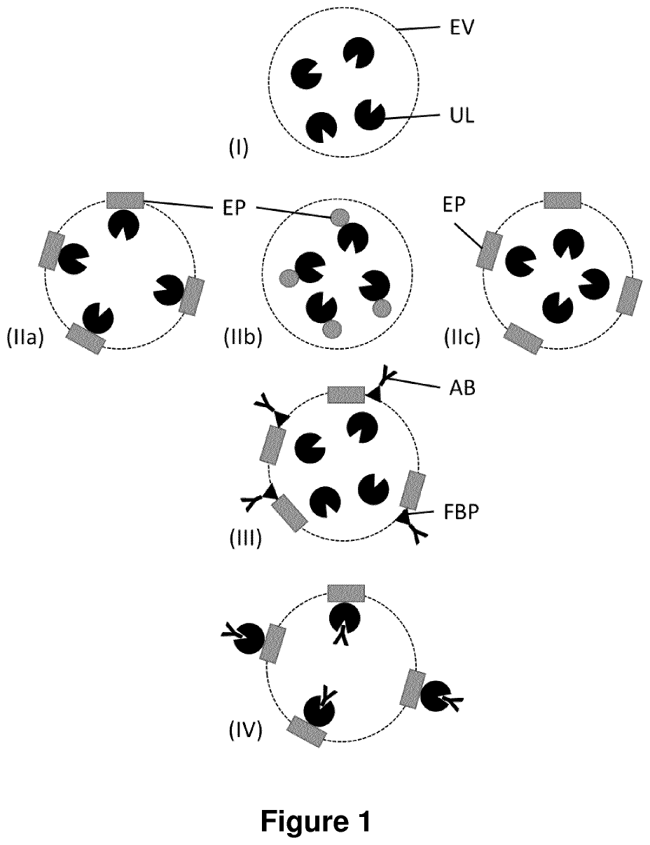

FIG. 2 shows GFP reduction in target cells following treatment with EVs comprising TRIM21 and anti-GFP antibodies. The greatest effect of GFP reduction was observed when EVs comprised a combination of ubiquitin ligase (TRIM21) and anti-GFP antibodies were co-incubated with GFP-expressing cells, whereas individual combinations of EV, antibodies or ubiquitin ligase had little effect.

FIG. 3 shows a dose response of NFkB pathway inhibition by the coincubation of cells with EVs comprising anti-NFkB antibody and TRIM21. An increase in concentration of loaded EVs further increases the inhibition response in the reporter cell line.

### DETAILED DESCRIPTION OF THE INVENTION

The inventors of the present invention have surprisingly discovered that extracellular vesicles (EVs), for instance exosomes, can be engineered to deliver ubiquitin ligases to enable ubiquitin-mediated target protein degradation. EVs are naturally occurring nano-sized vesicles secreted into the extracellular environment by EV-producing cells. EVs and in particular exosomes have been shown to be able to transport protein biologics, such as antibodies and decoy receptors, into target cells, enabling an entirely novel form of advanced biological therapeutics harnessing the properties of EVs in combination with the specificity of recombinant proteins. EVs provide several advantages over the conventional methods of biologics administration. For example, when biotherapeutics are delivered using EVs they are protected from degradation and are more stable; EVs constitute a multivalent drug delivery modality which may lead to enhanced efficacy; EVs may improve the pharmacokinetics and the pharmacodynamics of a protein biologic; EVs can be targeted to tissues and cells of interest; EVs may have inherent therapeutic effects reflecting their cellular origin; and, EVs also enable penetration of the blood-brain-barrier and improved CNS delivery.

The present invention thus relates to engineered EVs comprising at least a ubiquitin ligase but optionally also an antibody binding to a target protein to be degraded. Furthermore, the invention relates to various adjacent aspects as will be described in greater detail below, for instance polypeptide constructs which aid the loading and escape of EVs, polynucleotide constructs encoding for such polypeptide constructs, vectors and cells comprising such polynucleotide and/or polypeptide constructs, production methods, compositions comprising a plurality of such polypeptide-containing EVs, as well as medical applications of such EVs and pharmaceutical compositions containing such EVs. Consequently, the present invention provides for a highly modifiable, targetable, and modular delivery vehicle for a very complex biological system undeliverable by other means.

For convenience and clarity, certain terms employed herein are collected and described below. Unless otherwise defined, all technical and scientific terms used herein have the same meaning as commonly understood by one of ordinary skill in the art to which this invention belongs.

For convenience and clarity, certain terms employed herein are collected and described below. Unless otherwise defined, all technical and scientific terms used herein have the same meaning as commonly understood by one of ordinary skill in the art to which this invention belongs. For example, the ubiquitin ligases described in connection with the EVs is to be understood to be disclosed and relevant also in the context of the ubiquitin ligase-containing polypeptides and in the context of the pharmaceutical compositions comprising EVs (which in turn comprises such polypeptide constructs), or as expression products of the polynucleotide constructs as per the present invention. Furthermore, certain embodiments described in connection with certain aspects, for instance the administration routes of the EVs, as described in relation to aspects pertaining to treating certain medical indications, may naturally also be relevant in connection with other aspects and/or embodiment such as aspects/embodiments pertaining to the pharmaceutical compositions or the intracellular delivery methods of the present invention. As a general remark, the ubiquitin ligase, the antibodies, the exosomal sorting domains (interchangeably termed EV sorting domains or EV proteins or similar), the multimerization domains, the cleaving domains, endosomal escape domains and the targeting moieties, the cell sources, and all other aspects, embodiments, and alternatives in accordance with the present invention may be freely combined in any and all possible combinations without deviating from the scope and the gist of the invention. Furthermore, any polypeptide or polynucleotide or any polypeptide or polynucleotide sequences (amino acid sequences or nucleotide sequences, respectively) of the present invention may deviate considerably from the original polypeptides, polynucleotides and sequences as long as any given molecule retains the ability to carry out the technical effect associated therewith. As long as their biological properties are retained the polypeptide and/or polynucleotide sequences according to the present application may deviate with as much as 50% (calculated using for instance BLAST or ClustalW) as compared to the native sequence, although a sequence identity that is as high as possible is preferable. The combination (fusion) of e.g. at least one ubiquitin ligase and at least one exosomal sorting domain implies that certain segments of the respective polypeptides may be replaced and/or modified, meaning that the deviation from the native sequence may be considerable as long as the key properties are conserved. Similar reasoning thus naturally applies to the polynucleotide sequences encoding for such polypeptides.

The terms “extracellular vesicle” or “EV” or “exosome” shall be understood to relate to any type of vesicle that is, for instance, obtainable from a cell, for instance a microvesicle (e.g. any vesicle shed from the plasma membrane of a cell), an exosome (e.g. any vesicle derived from the endo-lysosomal pathway), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. Furthermore, the said terms shall also be understood to relate to lipoprotein particles, such as LDL, VLDL, HDL and chylomicrons, as well as extracellular vesicle mimics, cellular membrane vesicles obtained through membrane extrusion or other techniques, etc. Essentially, the present invention may relate to any type of lipid-based structure (with vesicular morphology or with any other type of suitable morphology) that can act as a delivery or transport vehicle for the ubiquitin ligase, and optionally an antibody. It will be clear to the skilled artisan that when describing medical and scientific uses and applications of the EVs, the present invention normally relates to a plurality of EVs, i.e. a population of EVs which may comprise thousands, millions, billions or even trillions of EVs, for instance concentrations such as 10⁵, 10⁸, 10¹⁰, 10¹¹, 10¹², 10¹³, 10¹⁴, 10¹⁵, 10¹⁸, 10²⁵, 10³⁰ per unit of volume. In the same vein, the term “population”, which may e.g. relate to an EV comprising various combinations or presentations of a ubiquitin ligase and antibody, shall be understood to encompass a plurality of entities which together constitute such a population. In other words, individual EVs when present in a plurality constitute an EV population. Thus, naturally, the present invention pertains both to individual EVs comprising a ubiquitin ligase or various combinations of a ubiquitin ligase and antibodies and populations comprising EVs which in turn comprise such ubiquitin ligase and/or antibody constructs, as will be clear to the skilled person.

The terms “antibody” and “mAb” and “Ab” as described herein is to be understood to include both antibodies in their entirety (i.e. whole antibodies) and any derivatives thereof with antigen-binding properties. Conventionally, an antibody refers to a glycoprotein comprising at least two heavy (H) chains and two light (L) chains inter-connected by disulfide bonds, or an antigen binding-portion thereof. Each heavy chain is comprised of a heavy chain variable region (abbreviated herein as VH) and a heavy chain constant region. Each light chain is comprised of a light chain variable region (abbreviated herein as V_{L}) and a light chain constant region. The variable regions of the heavy and light chains contain a binding domain that interacts with an antigen. The V_{H} and V_{L} regions can be further subdivided into regions of hypervariability, termed complementarity determining regions (CDR), interspersed with regions that are more conserved, termed framework regions (FR). Importantly, for the purposes of the present invention an antibody of interest preferably has an Fc domain to which the Fc binder can bind, in order to enable coating of the EV surface. Antibodies of use in the invention may be monoclonal antibodies (mAbs) or polyclonal antibodies, preferably mAbs. An antibody of use in the invention may be a chimeric antibody, a CDR-grafted antibody, a nanobody, a human or humanised antibody or any derivative thereof as long as it can be bound by the Fc binder protein comprised in the fusion proteins as per the present invention. The production of antibodies is outside of the scope of the present invention but typically both monoclonal and polyclonal antibodies are raised experimentally in non-human mammals such as goat, rabbit, rat or mouse, but suitable antibodies may also be the result of other production methodologies, e.g. the standard somatic cell hybridization technique of Kohler and Milstein. The preferred animal system for preparing hybridomas is the murine system. Hybridoma production in the mouse is a very well-established procedure and can be achieved using techniques well known in the art. An antibody of use in the invention may be a human antibody, humanized antibody, and/or any type of chimeric antibody. The term “human antibody”, as used herein, is intended to include antibodies having variable regions in which both the framework and CDR regions are derived from human germline immunoglobulin sequences. The human antibodies of use in the invention may include amino acid residues not encoded by human germline immunoglobulin sequences (e.g., mutations introduced by random or site-specific mutagenesis in vitro or by somatic mutation in vivo). The term “antibody derivatives” refers to any modified form of an antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications may be made within the human framework sequences. Antibodies in accordance with the present invention may include all isotypes and subtypes such as IgG, IgA, IgM, IgM, IgD, etc., and monomers, dimers, and oligomers thereof. Further, antibodies as per the present invention may have several functions when displayed on EVs: (1) the antibodies may as per the preferred embodiments of the present invention recognize and bind to antigens comprised in specific target biomolecules, which are meant to be degraded using the ubiquitin ligase systems as described herein; (2) antibodies may target specific cell types, tissues, and/or organs in order to re-direct distribution and optimize delivery of the EV-based therapeutics as described herein, (3) therapeutic antibodies that interact with a target antigen of interest can be efficiently delivered to tissues of interest using EVs (for instance to the CNS or to the brain), (4) multiplexed antibodies on the surface of EVs may be significantly better at binding target antigens, including target antigens which are meant to undergo ubiquitin ligase triggered degradation, (5) antibody-drug conjugates (ADCs) may be multiplexed on EVs to significantly enhance their therapeutic efficacy, and (6) coating of EVs with antibodies may reduce opsonization and/or immune-mediated clearance of EVs, which may in turn be important for therapeutic activity.

The terms “EV protein” and “EV polypeptide” and “exosomal polypeptide” and “exosomal protein” and similar are used interchangeably herein and shall be understood to relate to any polypeptide that can be utilized to transport a polypeptide construct (which typically comprises, in addition to the EV protein, an Fc binding polypeptide) to a suitable vesicular structure, i.e. to a suitable EV. More specifically, these terms shall be understood as comprising any polypeptide that enables transporting, trafficking or shuttling of a fusion protein construct to a vesicular structure, such as an EV. Examples of such exosomal polypeptides are for instance CD9, CD53, CD63, CD81, CD54, CD50, FLOT1, FLOT2, CD49d, CD71 (also known as the transferrin receptor) and its endosomal sorting domain, i.e. the transferrin receptor endosomal sorting domain, CD133, CD138 (syndecan-1), CD235a, ALIX, Syntenin-1, Syntenin-2, Lamp2b, syndecan-2, syndecan-3, syndecan-4, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, TSG101, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, other exosomal polypeptides, and any combinations thereof, but numerous other polypeptides capable of transporting a polypeptide construct to an EV are comprised within the scope of the present invention. Typically, in many embodiments of the present invention, at least one exosomal polypeptide is fused to at least one ubiquitin ligase and/or to essentially any Fc binding polypeptide, in order to form a fusion protein present in an EV. Such fusion proteins may also comprise various other components to optimize their function(s), including linkers, transmembrane domains, cytosolic domains, multimerization domains, etc. The proteins and polypeptides mentioned herein are preferable of human origin but may also be obtained from other mammals or non-mammals.

The terms “source cell” or “EV source cell” or “parental cell” or “cell source” or “EV-producing cell” or any other similar terminology shall be understood to relate to any type of cell that is capable of producing EVs, e.g. exosomes, normally under suitable cell culturing conditions, in vitro, ex vivo or in vivo. Such conditions may be suspension cell culture or adherent cell culture or any in other type of culturing system. Hollow-fiber bioreactors, shaking incubators and other types of bioreactors represent highly suitable cell culturing infrastructure and so does various bioreactors for suspension cells. The source cells per the present invention may be selected from a wide range of cells and cell lines, for instance mesenchymal stem or stromal cells or fibroblasts (obtainable from e.g. bone marrow, adipose tissue, Wharton's jelly, perinatal tissue, placenta, amnion, tooth buds, umbilical cord blood, skin tissue, etc.), amnion cells and more specifically amnion epithelial cells, optionally expressing various early markers, etc. Cell lines of particular interest include human umbilical cord endothelial cells (HUVECs), human embryonic kidney (HEK) cells, human amnion epithelial cells, endothelial cell lines such as microvascular or lymphatic endothelial cells, chondrocytes, MSCs, airway or alveolar epithelial cells, and various other non-limiting examples of cell sources. As abovementioned, MSCs may be obtainable from various sources, for instance bone marrow, adipose tissue, Wharton's jelly, perinatal tissue (e.g. amnion, amniotic membrane, amniotic fluid, chorion, placenta, umbilical cord, Wharton's jelly), tooth buds, umbilical cord blood, skin, etc. In general, EV-producing source cells may be either allogeneic, autologous, or even xenogeneic in nature to the patient to be treated, i.e. the cells may be from the patient himself or from an unrelated or related, matched or unmatched donor. The source cells of the present invention are preferably of human origin, especially when the subject to be treated is a human. However, other EV-producing cell sources are also within the scope of the present invention, for instance cells obtainable from other mammals, from rodents, or from any other suitable species or genus.

In a first aspect, the invention relates to EVs engineered to comprise a ubiquitin ligase. By using genetic engineering and overexpression strategies multiple copies of a ubiquitin ligase may be loaded into each and every vesicle, e.g. each and every exosome, which means that any population of EVs will comprise a large number of ubiquitin ligases. EVs provide for a unique means of transport for the ubiquitin ligase, especially within an in vivo setting. In addition, an EV provides a platform in which a variety of bioactive macromolecules can be associated and delivered, thus enabling transporting the ubiquitin ligase with additional components for its activity, including active targeting to specific tissues, immuno-evasion, bioactive DNA/RNA species, bioactive protein species (such as antibodies) and bioactive small molecules.

In one embodiment of the invention, the ubiquitin ligase comprised in the EV is an E3 ubiquitin ligase. E3 ubiquitin ligases are directly involved in the ubiquitination of their target and thus mediate protein-specific protein regulation. While it is preferred to load (associate) a single species of E3 ubiquitin ligase with an EV, it is possible to load several species of E3 ligase with an EV. The E3 ubiquitin ligase may be selected from any one of the ubiquitin ligases of the following group: AFF4, AMFR, ANAPC11, ANKIB1, AREL1, ARIH1, ARIH2, BARD1, BFAR, BIRC2, BIRC3, BIRC7, BIRC8, BMI1, BRAP, BRCA1, CBL, CBLB, CBLC, CBLL1, CCDC36, CCNB11P1, CGRRF1, CHFR, CNOT4, CUL9, CYHR1, DCST1, DTX1, DTX2, DTX3, DTX3L, DTX4, DZIP3, E4F1, FANCL, G2E3, HACE1, HECTD1, HECTD2, HECTD3, HECTD4, HECW1, HECW2, HERC1, HERC2, HERC3, HERC4, HERC5, HERC6, HLTF, HUWE1, IRF2BP1, IRF2BP2, IRF2BPL, Itch, KCMF1, KMT2C, KMT2D, LNX1, LNX2, LONRF1, LONRF2, LONRF3, LRSAM1, LTN1, MAEA, MAP3K1, MARCH1, MARCH10, MARCH11, MARCH2, MARCH3, MARCH4, MARCH5, MARCH6, MARCH7, MARCH8, MARCH9, Mdm2, MDM4, MECOM, MEX3A, MEX3B, MEX3C, MEX3D, MGRN1, MIB1, MIB2, MID1, MID2, MKRN1, MKRN2, MKRN3, MKRN4P, MNAT1, MSL2, MUL1, MYCBP2, MYLIP, NEDD4, NEDD4L, NEURL1, NEURL1B, NEURL3, NFX1, NFXL1, NHLRC1, NOSIP, NSMCE1, PARK2, PCGF1, PCGF2, PCGF3, PCGF5, PCGF6, PDZRN3, PDZRN4, PELI1, PELI2, PELI3, PEX10, PEX12, PEX2, PHF7, PHRF1, PJA1, PJA2, PLAG1, PLAGL1, PML, PPIL2, PRPF19, RAD18, RAG1, RAPSN, RBBP6, RBCK1, RBX1, RC3H1, RC3H2, RCHY1, RFFL, RFPL1, RFPL2, RFPL3, RFPL4A, RFPL4AL1, RFPL4B, RFWD2, RFWD3, RING1, RLF, RLIM, RMND5A, RMND5B, RNF10, RNF103, RNF11, RNF111, RNF112, RNF113A, RNF113B, RNF114, RNF115, RNF121, RNF122, NF123, RNF125, RNF126, RNF128, RNF13, RNF130, RNF133, RNF135, RNF138, RNF139, RNF14, RNF141, RNF144A, RNF144B, RNF145, RNF146, RNF148, RNF149, RNF150, RNF151, RNF152, RNF157, RNF165, RNF166, RNF167, RNF168, RNF169, RNF17, RNF170, RNF175, RNF180, RNF181, RNF182, RNF183, RNF185, RNF186, RNF187, RNF19A, RNF19B, RNF2, RNF20, RNF207, RNF208, RNF212, RNF212B, RNF213, RNF214, RNF215, RNF216, RNF217, RNF219, RNF220, RNF222, RNF223, RNF224, RNF225, RNF24, RNF25, RNF26, RNF31, RNF32, RNF34, RNF38, RNF39, RNF4, RNF40, RNF41, RNF43, RNF44, RNF5, RNF6, RNF7, RNF8, RNFT1, RNFT2, RSPRY1, SCAF11, SH3RF1, SH3RF2, SH3RF3, SHPRH, SIAH1, SIAH2, SIAH3, SMURF1, SMURF2, STUB1, SYVN1, TMEM129, Topors, TRAF2, TRAF3, TRAF4, TRAF5, TRAF6, TRAF7, TRAIP, TRIM10, TRIM11, TRIM13, TRIM15, TRIM17, TRIM2, TRIM21, TRIM22, TRIM23, TRIM24, TRIM25, TRIM26, TRIM27, TRIM28, TRIM3, TRIM31, TRIM32, TRIM33, TRIM34, TRIM35, TRIM36, TRIM37, TRIM38, TRIM39, TRIM4, TRIM40, TRIM41, TRIM42, TRIM43, TRIM43B, TRIM45, TRIM46, TRIM47, TRIM48, TRIM49, TRIM49B, TRIM49C, TRIM49D1, TRIM5, TRIM50, TRIM51, TRIM52, TRIM54, TRIM55, TRIM56, TRIM58, TRIM59, TRIM6, TRIM60, TRIM61, TRIM62, TRIM63, TRIM64, TRIM64B, TRIM64C, TRIM65, TRIM67, TRIM68, TRIM69, TRIM7, TRIM71, TRIM72, TRIM73, TRIM74, TRIM75P, TRIM77, TRIM8, TRIM9, TRIML1, TRIML2, TRIP12, TTC3, UBE3A, UBE3B, UBE3C, UBE3D, UBE4A, UBE4B, UBOX5, UBR1, UBR2, UBR3, UBR4, UBR5, UBR7, UHRF1, UHRF2, UNK, UNKL, VPS11, VPS18, VPS41, VPS8, WDR59, WDSUB1, WWP1, WWP2, XIAP, ZBTB12, ZFP91, ZFPL1, ZNF280A, ZNF341, ZNF511, ZNF521, ZNF598, ZNF645, ZNRF1, ZNRF2, ZNRF3, ZNRF4, Zswim2, ZXDC. The ligases of the present invention may be overexpressed by the source cell producing the EVs, i.e. the EV-producing cells may be genetically engineered to express in large quantities the ligases herein, in order to create engineered, modified EVs.

In a preferred embodiment, the E3 ubiquitin ligase is a TRIM ligase, preferably a TRIM21 ligase or any domain, region or functional derivative thereof. Many TRIM ligases, for instance the TRIM21 ligase, has the ability to bind Fc domains, which enables a dual use of the TRIM ligases as (1) antibody binders in themselves which can be used to coat EVs with antibodies, and (2) as mediators of ubiquitin-induced target protein degradation via the enzymatic ubiquitin ligase activity of the TRIM ligases, which results in proteasome-mediated target protein degradation.

Particularly advantageous fusion proteins as per the present invention may comprise the EV proteins CD63, CD81, CD9, Lamp2, syndecan, and syntenin, fused to at least one copy of a ubiquitin ligase. Designing the ubiquitin ligase (such as any TRIM ligase, for instance TRIM21) to be present within the lumen of EVs is sometimes preferable in order to prevent the interaction of the Fc-binding domain of TRIM21 with naturally occurring Fc domains within serum. Consequently, in a preferred embodiment the ubiquitin ligase is present essentially inside the EV, i.e. the majority of the ubiquitin ligase is present within the vesicle. This can be achieved by fusing the ubiquitin ligase to a suitable EV protein which is present inside the lumen of EVs or which can be used as a fusion partner to locate the ubiquitin ligase within the EV. Examples of such EV proteins include suitable domains and parts of several tetraspanins for instance CD63, but also soluble EV proteins such as syntenin, syndecan, or Alix. As TRIM21 typically binds to Fc domains, exposure to an environment like serum may result in it binding environmental antibodies, rendering the targeted degradative capacity less effective. However, in alternative embodiments, TRIM21 may be present within the membrane of the EV or on the surface of the EV.

In an additional aspect, the EV would further comprise an antibody. Such antibodies may be loaded into or onto EVs using the native affinity of TRIM ligases, for instance TRIM21, for the Fc domain of antibodies. However, antibodies may also be loaded into EVs by other means, such as fusion to EV proteins or by introducing Fc binding polypeptides into EVs. In addition to antibodies, any type of polypeptide and/or protein may be fused to an Fc domain, in order to enable a ubiquitin ligase such as TRIM to interact with it. Such proteins fused to an Fc domain could also be for protein targeting, including nanobodys, affibodys, DARPins, Fabs, scFvs, VLs, VHs, monobodies, anticailins as well as any other protein fused with an FC domain. Naturally, one single EV may comprise with more than one type of Fc domain-containing protein, for instance two different types of antibodies binding to different antigens, in order to enable, for instance, simultaneous targeting. One single EV may also, as is typically the case, comprise a substantial plurality of one single type of Fc domain-containing protein, such as one type of monoclonal antibody. Various combinations of targeting antibodies, therapeutic antibodies, antibody-drug conjugates (ADCs), and antibodies for reducing opsonization and/or immune cell-mediated clearance constitute preferred embodiments of the present invention. In advantageous embodiments, the EVs according to the present invention comprise a plurality of proteins comprising an Fc domain. Thus, the present invention may house at least 10 proteins comprising an Fc domain, preferably at least 50 proteins comprising an Fc domain, even more preferably at least 100 proteins comprising an Fc domain. Such proteins may be copies of the same protein or more than one protein. In a one embodiment, antibodies associated with an EV have a specific target located either intracellularly, extracellularly and/or located in a transmembrane localization. Preferred is an intracellular target, as it would enable the usage of the host cells ubiquitination and degradative pathways, but extracellular targets are also of strong therapeutic interest. Advantageously, an EV may comprise more than one type of antibody, in which a subset interact with an intracellular target for ubiquitination and another with an extracellular target, facilitating targeting to specific tissues or cell types. It is conceived that mixed population of antibodies may be present only within the EV lumen, only on the exterior of the EV, or present on both the inside and outside of the EV. An example may be antibodies associated with an intracellular target for targeted degradation are loaded within the lumen and antibodies associated with tissue targeting are displayed on the EV surface.

In a further embodiment, the antibodies attached to the external surface of the EV are attached by non-covalent interactions with the EV surface, for instance by interaction with specific antibody-binding proteins, for example the Z-domain of Protein A or by any Fc receptor, for instance FCGRI (CD64), FCGR2A (CD32A), FCGR2B (CD32B), FCGR2C (CD32C), FCGR3A (CD16A), FCGR3B (CD16B), FCAMR, FCERA, FCAR, or mouse FCGRI, FCGRIIB, FCGRIII, FCGRIV, and/or FCGRn. Alternatively, EV-antibody interaction could be facilitated by covalent bonding, for example, appropriately modified antibodies bearing the correct ligand attached to EVs bearing appropriate chemical receptors, for example for formation of succinimidyl esters or linkage by azide-alkyne cycloaddition. Alternatively, as abovementioned, the exosomal polypeptides mentioned herein may be used to either directly load an antibody into and/or onto an EV, but exosomal polypeptides may also be used to create fusion polypeptides together with Fc binding polypeptides which can then in turn bind to the Fc domains of antibodies. As above-mentioned, the Fc domain of the antibody may be bound by an Fc-binding polypeptide comprised in the EV and/or in fact by the ubiquitin ligase itself, using for instance the antibody-binding capacity of e.g. TRIM21. This interaction between the ubiquitin ligase and the Fc domain of an antibody (typically an IgG antibody) can occur in a variety of environments, such as in the intracellular EV progenitor cell environment, during any EV production process, within an environment containing serum such as an extracellular environment and within an intracellular environment containing the target of the antibody.

In further embodiments of the present invention, the EV may further comprise a ubiquitin-conjugating enzyme (such as an E2 ubiquitin-conjugating enzyme) and/or a ubiquitin-activating enzyme (such as an E1 ubiquitin-activating enzyme). By engineering these additional components of the ubiquitin ligase system into an EV the entire ubiquitination machinery is comprised in a single vesicle, meaning that the degradation of the target protein is highly efficient.

In a further aspect, the present invention relates to a method for degrading a target protein, comprising the steps of (i) providing a target antigen bound by an antibody and (ii) using an EV to deliver a ubiquitin ligase into proximity of the antibody. Said method for degrading a target protein may be an in-vitro or non-therapeutic method.

In yet another aspect, the invention relates to a method to degrade a target protein by the delivery of a ubiquitin ligase and an antibody using an EV. Degradation of the target protein is induced by its ubiquitination by the ubiquitin ligase delivered by the EV. Recognition of the target protein (antigen) is facilitated by the appropriate antibody, which in essence acts as an adaptor molecule between the target protein/antigen and the ubiquitin ligase. While the preferred method for protein degradation is by the EV-mediated co-delivery of ubiquitin ligase and antibody in the same EV or at least in the same EV population, their delivery may be separate events. Furthermore, delivery of, and antigen-antibody interaction may be an entire independent event from ubiquitin ligase delivery. Thus in a preferred subsequent embodiment, both the antibody and ubiquitin ligase are both delivered by an EV, and preferably by the same EV.

In further aspects, the present invention also pertains to inventive polynucleotide and polypeptide constructs. The polynucleotide constructs as per the present invention typically comprise nucleotide stretches encoding for at least one ubiquitin ligase and at least one exosomal polypeptide. A non-limiting example would be a polynucleotide construct encoding for a ubiquitin ligase, preferably an E3 ubiquitin ligase, more preferably the ubiquitin ligase TRIM21, and any exosomal polypeptide. Preferred exosomal polypeptides include CD81, syntenin, syndecan, CD63, Alix, the transferrin receptor, the endosomal domain of the transferrin receptor, etc. Thus, the present invention naturally also relates to the corresponding polypeptide constructs, i.e. polypeptide constructs comprising at least one ubiquitin ligase polypeptide and at least one exosomal polypeptide. Furthermore, the present invention also pertains to EV-producing cells (typically cells present in the form of cell culture but also individual cells as such) comprising the above-mentioned polynucleotide construct(s) and/or the above-mentioned polypeptide(s).

In yet another embodiment, the EVs of the present invention further comprise at least one targeting moiety. Typically, the targeting moiety is present on the surface of the EV (i.e. protruding from the EV membrane into the extravesicular environment) in order to facilitate reaching the correct tissue or cell type in vivo and/or in vitro. The EVs may also further comprise elements which enhance the loading of the ubiquitin ligase into EVs and their functioning within recipient cells. Such elements include multimerization domains which enhance EV loading, endosomal escape domains which enhance the efficiency of escape of EV cargo following uptake, release domains which improve the solubility of EV intraluminal cargo. The polypeptide constructs may comprise the ubiquitin ligase, exosomal protein, targeting moiety or enhancing elements as a single entity, or alternatively these different domains may be present in separate polypeptide constructs, which may be encoded by single or multiple polynucleotide constructs.

In yet another aspect, the present invention relates to a vector comprising the polynucleotide constructs as described herein. Such vectors may be used to create the EVs loaded with the ubiquitin ligase and optionally the antibody, but they may also be used as therapeutics in their own right. Non-limiting examples of vectors carrying the polynucleotide constructs as per the present invention include any linear or circularized polynucleotide, a circular DNA or RNA polynucleotide, a plasmid, a mini-circle, a virus such as an adenovirus or a lentivirus, an adeno-associated virus, a capsid-free virus, a viral genome, an mRNA, and/or a modified mRNA.

The introduction of suitable polynucleotide constructs into a source cell (typically in a cell culture comprising a suitable EV-producing cell type for production of EVs) may be achieved using a variety of conventional techniques, such as transfection, virus-mediated transformation, electroporation, etc. Transfection may be carried out using conventional transfection reagents such as liposomes, CPPs, cationic lipids or polymers, calcium phosphates, dendrimers, etc. Virus-mediated transduction is also a highly suitable methodology, and may be carried out using conventional virus vectors such as adenoviral or lentiviral vectors. Virus-mediated transduction and non-viral methods for stable transfection is particularly relevant when creating stable cells and cell lines for cell banking for research and development purposes, i.e. the creation of master cell banks (MCBs) and working cell banks (WCBs) of EV-producing cell sources. The polynucleotide constructs and/or vectors of the present invention can be used to overexpress the ubiquitin ligase in the producer cells, thereby increasing the levels of ubiquitin ligase introduced into the EV produced by the producer cells. This advantageously results in genetically engineered EVs loaded with higher levels of ubiquitin ligase, which are therefore more therapeutically potent meaning that fewer EVs per dose are required to achieve the same therapeutic effect.

In yet another aspect, the present invention relates to a method for producing an EVs as per the present invention. Said methods may comprise the steps of (i) introducing into an EV-producing cell at least one polynucleotide construct as described herein, (ii) expressing in the EV-producing cell at least one polypeptide construct encoded for by the at least one polynucleotide construct; and (iii) obtaining EVs produced by said EV-producing cell. The EV production scale and timeline will be heavily dependent on the EV-producing cell or cell line and may thus be adapted accordingly by a person skilled in the art. The methods as per the present invention may further comprise an EV purification step. In the cases where an antibody or any other type of Fc-containing protein is to be attached to an Fc-binding polypeptide on the EV surface the EVs may also in one step by co-incubated with the Fc domain-containing protein (typically an antibody) in question. This can take place immediately after EV production by the EV-producing source cell, or it may take place once EVs have been purified one or more times using a variety of purification and isolation techniques. EVs may be purified through one or more procedures selected from a group of techniques comprising liquid chromatography (LC), ion-exchange LC, size-exclusion LC, bead-elute LC, high-performance liquid chromatography (HPLC), spin filtration, tangential flow filtration (TFF), hollow fiber filtration, centrifugation, immunoprecipitation, flow field fractionation, dialysis, microfluidic-based separation, etc., or any combination thereof.

In an advantageous embodiment, the purification of the EVs is carried out using a sequential combination of filtration (preferably ultrafiltration (UF), tangential flow filtration or hollow fiber filtration) and size-exclusion or ion-exchange liquid chromatography (LC) or bead-elute LC. This combination of purification steps results in optimized purification, which in turn leads to superior therapeutic activity.

In a further embodiment, the methods may include introducing an additional polynucleotide construct into an EV-producing cell, wherein the additional construct encodes for an Fc-binding polypeptide which is thus loaded into the EVs for binding to antibodies and/or other proteins comprising Fc domains. Production of EVs from such producer cells are advantageous, as they comprise an Fc-binding component on their surface for the binding of Fc-containing polypeptides such as an antibody, thereby enhancing EV targeting. Advantageously, the EVs as per the present invention may thus be coated with a plurality of proteins comprising an Fc domain, through interaction between the Fc binder that is engineered into the EV and at least one protein comprising an Fc domain. The interaction between the Fc binder and the protein, typically an antibody which comprises an Fc domain, is typically based on primarily non-covalent interactions. Naturally, one single EV may be coated with more than one type of Fc domain-containing protein, for instance two different types of antibodies binding to different antigens, in order to enable, for instance, simultaneous targeting and therapeutic antigen binding for subsequent ubuiqitin-mediated degradation. One single EV may also, as is typically the case, comprise a substantial plurality of one single type of Fc domain-containing protein, such as one type of monoclonal antibody with a particular target. As above-mentioned, the antibodies may be introduced into the EVs by exogenous loading or endogenous production by the producer cell. Typically, exogenous addition of antibodies or antibody derivates is performed following the production of EVs from the producer cell. Alternatively, antibodies or antibody derivatives may be produced endogenously in the EV producer cell, and associate with the EV intracellularly.

In yet another aspect, the present invention pertains to pharmaceutical compositions comprising EVs, normally in the form of populations of EVs, as per the present invention. Typically, the pharmaceutical compositions as per the present invention comprise one type of therapeutic EV (i.e. a population of EVs comprising a certain type of fusion protein comprising an exosomal protein and a ubiquitin ligase, and optionally comprising one or more types of antibodies) formulated with at least one pharmaceutically acceptable excipient, but more than one type of EV population may naturally be comprised in a pharmaceutical composition, for instance in cases where a combinatorial protein-degradation treatment is desirable. Naturally however, as above-mentioned, a single EV or a single population of EVs may comprise more than ubiquitin ligase and more than one Fc-containing protein (e.g. more than one antibody). The at least one pharmaceutically acceptable excipient may be selected from the group comprising any pharmaceutically acceptable material, composition or vehicle, for instance a solid or liquid filler, a diluent, an excipient, a carrier, a solvent or an encapsulating material, which may be involved in e.g. suspending, maintaining the activity of or carrying or transporting the EV population from one organ, or portion of the body, to another organ, or portion of the body (e.g. from the blood to any tissue and/or organ and/or body part of interest). In a further embodiment, the pharmaceutical composition may further comprise at least one antibody. The composition may contain a single antibody species or a several antibody species.

In yet another aspect, the present invention relates to EVs as per the present invention for use in medicine. Naturally, when an EV comprising an exosomal protein and a ubiquitin ligase, and one or more types of antibodies is used in medicine, it is in fact normally a population of EVs that is being used. The dose of EVs administered to a patient will depend on the number of e.g. ubiquitin ligase containing EVs, antibodies of interest that associate with the EV, the disease or the symptoms to be treated or alleviated, the administration route, the pharmacological action of the therapeutic protein itself, the inherent properties of the EVs, the presence of any targeting antibodies or other targeting entities, as well as various other parameters of relevance known to a skilled person.

The EVs and the EV populations thereof as per the present invention may thus be used for prophylactic and/or therapeutic purposes, e.g. for use in the prophylaxis and/or treatment and/or alleviation of various diseases and disorders. A non-limiting sample of diseases wherein the EVs as per the present invention may be applied comprises Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barré syndrome, acute myocardial infarction, ARDS, sepsis, meningitis, encephalitis, liver failure, non-alcoholic steatohepatitis (NASH), kidney failure, heart failure or any acute or chronic organ failure and the associated underlying etiology, graft-vs-host disease, Duchenne muscular dystrophy and other muscular dystrophies, lysosomal storage diseases such as Gaucher disease, Fabry's disease, MPS I, II (Hunter syndrome), and III, Niemann-Pick disease, Pompe disease, etc., neurodegenerative diseases including Alzheimer's disease, Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral, Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sezary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor.

The EVs as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the antibody or the EV population as such.

It shall be understood that the above described exemplifying aspects, embodiments, alternatives, and variants can be modified without departing from the scope of the invention. The invention will now be further exemplified with the enclosed examples, which naturally also can be modified considerably without departing from the scope and the gist of the invention.

### EXAMPLE 1

### Intracellular Uptake and GFP Depletion by TRIM21-antiGFP-Ab-EVs

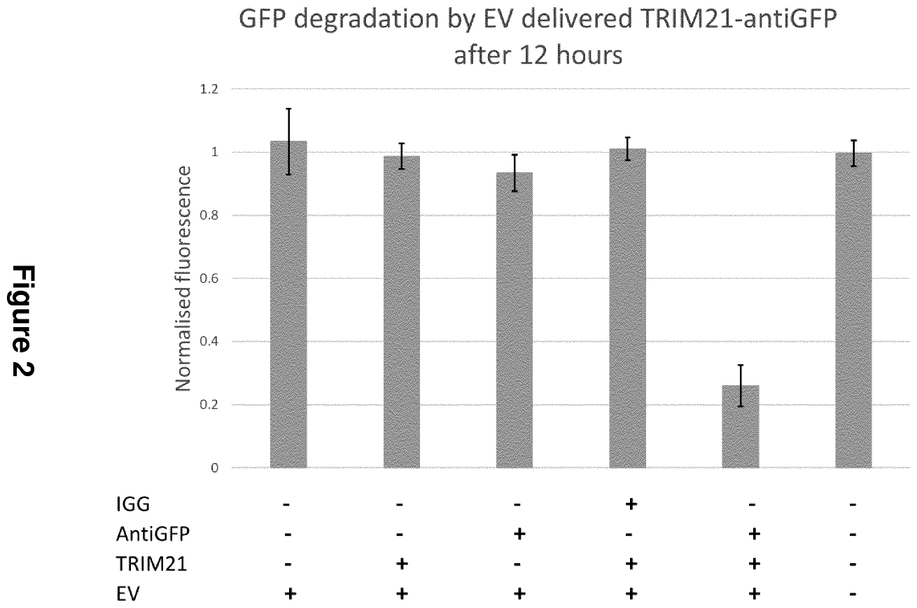

EVs were isolated from the conditioned medium of Wharton's jelly-derived MSCs (stably expressing the fusion CD81-intein-TRIM21 (the intein modality acts a release mechanism, separating TRIM21 frim CD81 following EV-loading) and the Fc-binder CD63-ZZ) using ultrafiltration and size exclusion chromatography. To load the EVs, 4×10\hat{ }11 EVs were incubated in 400 μl for 16 hours (overnight) with total 3 μg anti-GFP IgGs [abcam (ab1218) Anti-GFP antibody [9F9.F9]]. To investigate whether EVs comprising TRIM21 and Fc-binding can be employed for intracellular depletion of GFP, GFP-expressing Huh7 cells were plated in 48 well plates at 30,000 cells per well and co-incubated with 2.4×10\hat{ }10 EVs for 12 hours (overnight). Cells were incubated for 2 hours at 37° C. and 5% CO₂ in a humidified atmosphere before they were trypsinized and analyzed by flow cytometry as shown in FIG. 2. The data demonstrate that only the combination of EV, TRIM21 and anti-GFP antibody results in a significant depletion of the GFP signal.

### EXAMPLE 2

### Dose Response of NFkB Depletion By TRIM21-antiNFkB-Ab-EVs

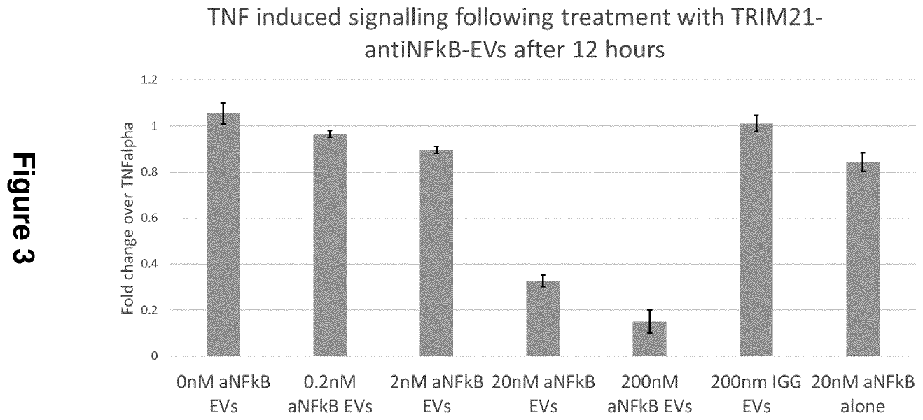

Simialr to Example 1, EVs were isolated from the conditioned medium of Wharton's jelly-derived MSCs (stably expressing the fusion CD81-intein-TRIM21 and the Fc-binder CD63-ZZ) using ultrafiltration and size exclusion chromatography. To load the EVs, 4×10A11 EVs were incubated in 400 μl for 16 hours (overnight) with total 3 μg anti-NFkB IgGs [abcam: (ab32360) Anti-NFkB p105/p50 antibody [E381]]. To investigate whether EVs comprising TRIM21 and Fc-binding can be employed for intracellular depletion of NFkB in a dose dependent manner, the reporter cell line of Ken293 stably expressing NFkB-luciferase cells were plated in 48 well plates at 30,000 cells per well and co-incubated with 2.4×10A10 EVs and 5 ng/ml hTNF-alpha. After 12 hours of treatment the luciferase activity was measured. FIG. 3 shows the normalised luciferase levels, demonstrating successful inhibition when the anti-NFkB-ab is delivered together with TRIM21 EVs, in a dose dependent manner.

1. A genetically engineered extracellular vesicle (EV) comprising a TRIM ligase or a domain or region thereof.

2. The EV according to claim 1, wherein the TRIM ligase or a domain or region thereof is a TRIM21 ligase or a domain or region thereof.

3. The EV according to claim 1, wherein the TRIM ligase is fused to an exosomal protein.

4. The EV according to claim 1, wherein the EV further comprises at least one antibody.

5. The EV according to claim 4, wherein the antibody is comprised in a fusion protein with an exosomal protein.

6. The EV according to claim 4, wherein the Fc domain of the antibody is bound by an Fc-binding polypeptide comprised in the EV and/or by the Fc binding region of the TRIM ligase.

7. The EV according to claim 4, wherein the target protein of the at least one antibody is degraded through the action of the TRIM ligase.

8. The EV according to claim 1, wherein the EV further comprises a ubiquitin-conjugating enzyme and/or a ubiquitin-activating enzyme.

9. A method for degrading a target protein, comprising the steps of (i) allowing an antibody to bind its antigen and (ii) delivering a ubiquitin ligase into proximity of the antibody with the aid of an EV.

10. The method according to claim 9, wherein the antibody is also delivered by an EV, optionally the same EV.

11. The method according to claim 9, wherein the target antigen is an intracellular target.

12. A polypeptide construct comprising an exosomal protein and a TRIM21 ligase.

13. A polynucleotide construct encoding for a polypeptide construct according to claim 12.

14. A vector comprising the polynucleotide construct according to claim 13.

15. The vector according to claim 14, wherein the vector is selected from the group comprising a linear or circularized polynucleotide, a circular DNA or RNA polynucleotide, a plasmid, a mini-circle, a virus, an adeno-associated virus, a capsid-free virus, an mRNA, a modified mRNA, and/or a synthetic mRNA.

16. A method for producing a genetically engineered EV according to claim 1, said method comprising the steps of:
(i) introducing into an EV-producing cell at least one polynucleotide construct encoding for a polypeptide construct comprising an exosomal protein and a TRIM21 ligase;
(ii) expressing in the EV-producing cell at least one polypeptide construct encoded for by the at least one polynucleotide construct; and,
(iii) obtaining EVs produced by said EV-producing cell.

17. The method according to claim 16, further comprising introducing and expressing from another polynucleotide construct in the EV-producing cell a polypeptide construct comprising an Fc binding polypeptide.

18. The method according to claim 17, wherein the Fc binding polypeptide binds an antibody.

19. The method according to claim 18, wherein the antibody is exogenously added to the EV in solution or endogenously produced by the EV-producing cell.

20. A pharmaceutical composition comprising a population of genetically engineered EVs according to claim 18 and a pharmaceutically acceptable excipient or diluent.

21. The pharmaceutical composition according to claim 20, further comprising at least one antibody.

22. A genetically engineered EV according to claim 1 for use in medicine.
